(19) United States
(12) Patent Application Publication
(10) Pub. No.: US 2014/0148778 A1
(43) Pub. Date: May 29, 2014

(54) Tube Anchor Assembly

(71) Applicant: Andrew Levy, Bristol (GB)

(72) Inventor: Andrew Levy, Bristol (GB)

(21) Appl. No.: 13/880,537

(22) PCT Filed: Oct. 19, 2011
(86) PCT No.: PCT/GB2011/001503

§ 371 Date: Jun. 14, 2013

(30) Foreign Application Priority Data
Oct. 21, 2010 (GB) 1017792.1

(51) Int. Cl. A61M 25/02

(52) U.S. Cl. CPC A61M 25/02
USPC 604/500; 604/174

(57) ABSTRACT

A device for supporting a tube is disclosed, the device comprising a flexible base for securing to the skin of a patient and having a first major surface for opposing the skin of the patient and a second major surface opposite the first, the base comprising a channel extending therethrough, the channel opening in the first or the second major surface of the base. The channel may be straight or arcuate. The device finds particular use in holding tubes, drains, cannulae, catheters and the like at the skin of a subject.

The present invention relates to an assembly for anchoring tubes, in particular to an assembly for attaching to the skin of a subject to hold one or more tubes.

It is common practice for a subject to be provided with one or more tubes during a wide range of medical procedures. For example, the subject may be provided with fluids intravenously, in which fluids are provided to the subject through a suitable tube connected to a catheter extending within a vein of the subject, commonly a vein in their hand or arm. Fluids may be provided intra-arterially in a similar manner, commonly into an artery in the wrist, thigh or neck of the subject. Further, it is known to provide fluids, such as plain fluids or nutritional supplements, to a subject who is typically unable to swallow safely who is being intubated through their nose or mouth, or directly into the gut form the skin surface. Still further, it is known to drain gases or liquids that have collected in body cavities, such as the chest (pneumothorax or pleural effusion) or abdomen (ascites, pus or blood), bladder or renal tract (urine), the biliary tree (bile). It is also known to provide oxygen enriched air or gas mixtures containing drugs, such as anaesthetics and analgesics to a subject through tubes. In addition, it is known to provide fluids, such as analgesics and anaesthetics used for nerve blocks, such as epidurals, and infusions directly into tissues for fluid replacement, when intravenous access is limited or inappropriate. A particular problem with such procedures is the management of the tubes being used, in particular their support and retention in a manner that avoids them be improperly handled or removed, causing injury to the subject. There is a need for a system for anchoring such tubes, in particular to the subject themselves.

GB 1,471,609 discloses a holder for elongate articles, in particular a holder for attaching to a person and for holding one or more tubes. The device comprises a generally rectangular, flexible base having extending from one major surface thereof a plurality of ridges, each ridge having a plurality of recesses for holding a tube. The ridges are preferably spaced apart and preferably extend substantially parallel to one another. In use, a tube or the like is held by a recess in each ridge. The device is attached to the skin of a subject by a suitable adhesive.

U.S. Pat. No. 4,583,976 discloses a catheter support. The support comprises an anchoring pad for attaching to the skin of a subject with a suitable adhesive. The support further comprises a flexible strap, a portion of which is attached to the pad. The strap is provided with a fastener material on one side. In use, the pad is attached to the skin of the subject. A catheter is laid over the pad and the strap is extended over the catheter and fastened to the pad. The pad and/or the flexible strap may be provided with a channel therethrough for receiving the catheter tube.

U.S. Pat. No. 5,833,663 discloses a naso-gastric tube retainer. The retainer comprises a first portion for adhering to the nose of a subject and a second portion for adhering to a naso-gastric tube. The two portions are connected by a flexible, inelastic spine.

A similar device is disclosed in WO 93/16750, the device comprising a generally tubular holding clamp for holding an naso-gastric or intubation tube and a connecting member. The connecting member connects the clamp with an adhesive member for securing to the nose of a patient.

U.S. Pat. No. 6,231,548 is concerned with a device for securing a intravenous cannula or catheter. The device comprises a generally rectangular, flexible base having a retainer mounted on a major surface thereof and extending therefrom. The retainer comprises a body having a channel extending therethrough, with a slotted opening therein. In use a tube is inserted into the channel through the slot and the device moved along the tube until the body engages with a catheter or luer hub, once the catheter or cannula has been inserted into the patient.

US 2005/182368 discloses a medical tubing anchor. The anchor comprises a flexible base for adhering to the skin of a patient. The anchor further comprises a flexible anchor member extending from a major surface of the base and provided with a plurality of channels or grooves therein, into which tubes may be inserted to be held. A keeper, in particular a strap, is provided to retain the tubes within the anchor member and prevent their inadvertent removal.

US 2006/289011 is concerned with a resilient nasal intubation tube support. The support comprises a block of foam having a generally U-shaped channel formed therein for receiving an intubation tube. The channel is provided with an adhesive to hold the tube in place. The block is provided with adhesive for securing the support to the skin of a patient, in particular their forehead.

US 2007/142784 discloses an anchoring device for an intravenous catheter. The device comprises a membrane for securing to a patient, a platform and a retaining strap connected to the platform. The strap is attached to the platform by means of adjustable pull-tabs, permitting the strap to be adjusted to suit a range of different tubes. The strap is preferably elastomeric, so as to grip one or more tubes.

US 2008/294117 is concerned with a flexible tube coupler. The coupler comprises a base for securing to the skin of a patient. A plurality of clamping elements extend from a major surface of the base and are provided with arcuate surfaces to partially surround and grip a tube inserted therebetween.

US 2009/143741 discloses a device with locating guides for securing medical lines and tubes. The device comprises an anchor pad having a flexible retainer opening for receiving a medical line or tube. The anchor pad is secured to a base and held in position by a plurality of locating posts extending from the base. The base is attached to the skin of a patient by a suitable adhesive.

WO 93/04726 concerns a catheterization placement apparatus comprising a fixation device for securing the catheter to the abdomen of the patient to prevent inadvertent movement thereof during use.

A retainer for a catheter is disclosed in U.S. Pat. No. 5,499,976, the retainer comprising a flexible flattened pad having a tunnel extending transversely therethrough for receiving the catheter. The back of the retainer is provided with a slit for introducing the catheter into the tunnel.

A retainer clip for securing breathing devices is described and shown in WO 2008/148085, the clip having a base and a clamp portion formed from a pair of spaced apart clamp fingers defining a channel.

U.S. Pat. No. 5,496,283 discloses an apparatus for securing intravenous or intracavity medical tubing.

A profiled support or guide member for accommodating tubes for carrying medicaments or for wound drainage is disclosed in DE 20007798. The guide member has curved and straight grooves with an adhesive surface to hold the tubes.

U.S. Pat. No. 3,568,679 discloses a catheter placement unit for retaining a catheter in a desired place within a body.

U.S. Pat. No. 3,537,452 concerns a needle cover and bevel guard, having a tubular body with a flat base and a longitudinally slotted top surface.

An infusion needle support is described and shown in U.S. Pat. No. 2,727,512.

EP 1 408 075 is concerned with a tube seal adaptor for respiratory masks, in particular to form a seal around a tube extending between a respiratory mask and the face of a user.

The devices of the prior art are generally of a complex design, typically comprising two or more components, which must be assembled or formed together.

Further, in many cases the devices comprise clamps or supports extending from a base. Such projections can lead to the device being snagged or caught, for example on a patients clothing, leading to pain and possible disturbance of the tubes.

Accordingly, there is a need for an improved device for supporting tubes and lines on a patient or subject.

In a first aspect, the present invention provides a device for supporting a tube or line, the device comprising a flexible base for securing to the skin of a patient and having a first major surface for opposing the skin of the patient and a second major surface opposite the first, the base comprising a channel extending therethrough, the channel opening in the first or the second major surface of the base.

In the device of the present invention, the tube or line is held in a channel extending in the flexible base of the device and opening in the first major surface, that is the surface facing or contacting the skin of the subject, or the second major surface of the device, that is the major surface exposed when the base is attached to the skin of a patient. In this way, the device and the tube being retained may be arranged to protrude the minimum amount from the skin of the patient, reducing or eliminating the possibility that the tube and or the device is dislodged from the patient by either the patient themselves or someone attending the patient. Further, the device is simple to form and use, in particular as it may be formed with the base as the single component.

In one embodiment, the channel is formed with its opening in the second major surface of the base, that is the surface exposed when the device is secured to the skin of a patient. This device has the advantage that the tube may be removed from the channel and its position adjusted, without necessarily removing the device from the skin of the subject.

In an alternative embodiment, the channel opening is in the first major surface of the base. In the device of this embodiment, the tube is held in a channel in the first major surface of the base. In use, the opening of the channel is facing or adjacent the skin of the patient. A tube is first inserted into the channel, its position adjusted and then the device is secured to the skin of the patient. The device of this aspect of the invention offers the advantage that the tube is held in the channel and may not be removed from the device, without first removing the device from the patient.

The device of the present invention may be formed and used to hold any medical tube, needle, cable, wire or line (hereafter simply referred to as ‘tubes’). Such tubes include catheters, cannulae, naso-gastric and oro-gastric tubes, PEG (percutaneous endoscopic gastrostomy) tubes and percutaneous endoscopic jejunostomy tubes, tubes delivering drugs and fluids into tissues, such as epidural tubes, analgesic and insulin infusion, tubes for fluid delivery when direct intravenous access is inappropriate, intubation tubes, such as for supplying oxygen or oxygen-enriched air to the patient through their nose or mouth. Other tubes include drain tubes allowing gases, effusions, urine and other tissue collections to escape from the subject. Further, the device of the present invention may be formed and used to hold wires and cables, such as those used for temporary pacemakers or other nerve stimulators, and secure electrodes used for ECG monitoring, particularly during lengthy recordings in ambulatory patients.

The device comprises a base. The base is flexible. The base may be formed from any suitable flexible material. The material is sufficiently flexible to allow the base to conform to the contours of the skin of the subject and allow the skin of the patient to move and flex in substantially a normal manner without excessive restraint. Further, the material of the base is flexible to allow a tube to be inserted into and removed from the channel, while being sufficiently rigid to hold the tube within the channel.

Suitable materials for forming the base of the device are known in the art and are commercially available. Polymers are particularly preferred materials, as these are available commercially in medically acceptable grades. Suitable polymers include copolymers and homopolymers. Examples of suitable polymers include medical grade polyesters, halogenated polymers, such as polyvinyl chloride, and polyolefins, such as polyethylene, polypropylene and polybutylene.

In one embodiment, the polymer of the base is one that exhibits significant friction with the material of the tube to be held. In this way, the material of the base assists in retaining the tube within the channel in the base, preventing the tube from leaving the channel and/or sliding longitudinally within the channel. Preferably, the material of the base is such that the tube is held within the channel by sufficient friction between the material and the tube at the walls of the channel, while allowing the tube to slide if subjected to a force exceeding a threshold value. In this way, the tube may be held securely within the device during normal use, but slide within the channel if the tube is suddenly tugged or pulled, for example by the patient. This avoids any sudden force exerted from removing the device from the skin of the subject. Further, material of the base preferably acts to damp the effects of sudden forces applied to the tube, in turn reducing the risk that the tube is dislodged. For example, such damping may reduce the risk of a cannula, catheter or drain telescoping in and out through an opening in the subject, thereby reducing the risk of infection or of the tube being displaced or removed from the subject.

The base may be formed by any suitable technique. In the case of the base being formed from a polymer, the base is preferably moulded. Suitable moulding techniques are known in the art and include vacuum moulding and injection moulding. It is an advantage of the device of the present invention that it may be prepared in a simple manner using such well known techniques as moulding. In particular, the device may be formed as a single component, that is the base, in a single moulding step.

The base may have any suitable thickness. In order to fully accommodate a tube, the thickness of the base, at least in the region of the channel, is greater than the diameter of the tube for which the device is intended for use. In particular, the thickness of the base, at least in the region of the channel, is preferably from 1.05 to 3 times the diameter of the tube for which the device is intended, more preferably from 1.1 to 2, still more preferably from 1.15 to 1.5 times the diameter of the intended tube. In the portions of the base more distant from the channel, the thickness may be reduced, in particular to increase the flexibility of the base.

The base may have any suitable thickness, depending upon the material from which the base is formed and the tube to be accommodated within the channel, as noted above. Typically, the base will have a thickness in the range of from 0.5 mm to 15 mm, more preferably from 10 to 15 mm. As noted, the thickness of the base may depend upon the diameter of the tube, lead or needle to be held. Thus, nasogastric tubes typically have a diameter of about 3 mm. Typical IV infusion tubing is generally about 5 mm in diameter. The base to accommodate such tubes may therefore be from 3.5 to 6 mm in thickness, at least in the region of the channel. Teflon tubes used for the infusion of drugs and anaesthetics tend to be smaller in diameter, for example from 0.5 to 1.0 mm. The base to accommodate such tubes may be from 0.75 to 1.5 mm in thickness. Fine needles may have a diameter of about 0.3 mm, requiring a base of thickness from 0.5 to 1.0 mm.

In one embodiment, the base has a uniform thickness across its width and length. In a preferred embodiment, the base has its thickest portion in the region of the channel in the base, the base tapering to a lower thickness in the edge regions of the base. This has the advantage of making the edge regions of the base more flexible, and thus more adaptable to the contours of the skin of the patient, in turn allowing the patient increased mobility while having the device secured to their skin.

The base may be of any suitable shape. For example, the base may be rectangular. If rectangular or like shape, with corners, the corners of the base are preferably rounded. Preferably, the base is rounded in shape, having curved sides, with preferred shapes being generally circular or oval. Other shapes may also be contemplated. For example, the device may be provided for children with the base in ornate shapes, to amuse the child.

The base may have any suitable width and length. The length of the base as referred to herein is the dimension of the base parallel to the channel and thus determines the length of channel that can be provided therein. Preferably, the base is of a length that provides a channel that is significantly longer than the diameter of the tube to be held. In particular, the base, and hence the channel formed therein, has a length that is from 5 to 25 times the diameter of the tube, more preferably from 10 to 20 times the diameter of the tube.

The width of the base, that is the dimension perpendicular to the channel in the base, is preferably selected to provide the base with stability once adhered to the skin of a patient. The ratio of the length of the base to the width may be in the range of from 1:0.25 to 1:2.5, more preferably from 1:0.5 to 1:1.5.

The base may be formed as a single component. This is particularly preferred in embodiments in which the channel of the device is substantially linear and non-curved. Alternatively, the base may comprise two or more sections, joined by a flexible member, such as a flexible layer or substrate extending between adjacent portions of the base to provide a flexible union or joint therebetween. The flexible layer or substrate is disposed on the base on the opposing surface to the surface having the channel opening therein. The base may be divided into the two or more portions along the line of the channel therein, thus allowing the base to flex about the length of the channel. This arrangement is particularly preferred in embodiments where the channel is curved, thus allowing the base to flex freely along the line of the channel to allow the tube to be inserted and removed more readily.

The flexible layer or substrate, if present, may be formed from any suitable material. Preferred materials are flexible polymers, as mentioned hereinbefore. The flexible layer may have any suitable thickness, preferably in the range of from 0.1 to 2.0 mm.

The flexible layer may extend partially across the adjacent portions of the base, so as to form a union or junction therebetween. Alternatively, the flexible layer may be coterminous with the surface of the base portions. In one embodiment, the flexible layer extends beyond the edges of the base, to increase the comfort of the subject when wearing the device and reduce abrasion to the skin of the subject at the edges of the base.

In one embodiment, the base is sufficiently wide to provide a portion of the exposed or second major surface that may be used to display information, such as notes to be written onto the surface by a medical practitioner, devices, images or the like.

In use, the base is secured to the patient, such that the first major surface of the base opposes or contacts the skin of the patient. Any suitable means may be used to secure the base to the skin of the patient. For example, medically acceptable adhesive tape may be used. In a particularly preferred embodiment, the first major surface of the base is provided with a layer of adhesive thereon, for securing the base to the skin of the patient. Suitable adhesives for contacting the skin of a patient are known in the art and are commercially available. The layer of adhesive may be provided with a layer or sheet of protective material, which is easily removed to expose the adhesive for use. Such arrangements are well known in the art.

The device comprises a channel extending in the base for holding a tube. The channel is open along its length with the elongate opening extending in the first or second major surface of the device. The device preferably comprises a single channel in the base. However, the device may comprise two or more channels of the same or different diameter, to accommodate tubes. The two or more channels may open in the same or different major surfaces of the base. If two or more channels are present, they preferably extend substantially parallel to each other.

In use, the channel holds a tube so as to locate the tube when the device is adhered to the skin of a patient. As the base of the device is made from a flexible material, it is not necessary to provide an adhesive or other means to hold the tube in the channel, as is required in certain prior art devices. Rather, the flexible material of the base allows the tube to be held within the channel. Preferably, the channel is arranged to grip the tube, so that the tube is not easily removed from the channel and is prevented from moving longitudinally within the channel. The channel preferably extends within the base from one edge of the base to the opposite edge, with the ends of the channel opening at the respective edges of the base.

The base may be formed to provide the channel to be close to or adjacent the skin of the subject, when in use. Alternatively, the base may be formed to provide a channel that is distanced from the skin of the subject, when in use. In this way, the proximal and distal ends of the tube extending from the ends of the channel have a reduced tendency to rub and abrade the skin of the subject.

In one embodiment, the channel is substantially straight or linear. In an alternative embodiment, the channel is curved or arcuate. It has been found that providing a curved channel in the base aids in holding and gripping a tube within the channel.

More generally, it has been found that a curved or arcuate channel provides improved grip on a tube disposed within the channel. Accordingly, in a further aspect, the present invention provides a device for supporting a tube or line, the device comprising a base for securing to the skin of a patient; and an arcuate channel for accommodating a tube or line to be supported. The channel may be formed in a supporting member attached to the base. More preferably, the channel is formed in the base, as herein described.

The channel may extend in any suitable curved pattern, sufficient to grip the tube to be held. The curve may consist of a single arc or may comprise a plurality of arcs, that is be a compound curve. In one preferred embodiment, the channel extends between its open ends in a compound curve that extends to one side of a line joining the open ends. Alternatively, the channel may extend in a curved pattern to both sides of the said line. The curve of the channel should be such as to grip a tube within the channel, without kinking or blocking the tube, when the device is in situ on a patient.

The channel may have straight sides, between which the tube is held, that is substantially linear sides extending from the opening of the channel to the base of the channel. In this case, the channel is sized to provide an interference fit of the tube within the channel, such that the tube is held by the flexible channel walls. More preferably, the sides of the channel are curved between the base of the channel and the channel opening, to more closely accommodate the tube. In this way, the channel contacts and holds a major portion of the surface of the tube. The base of the channel may be substantially flat, but is preferably curved, to more closely accommodate the outer profile of the tube to be held. Preferably, the channel is substantially circular in cross-section, with the inner diameter of the channel being substantially the same but no greater than the diameter of the tube to be held and sized such that the tube is held by friction within the channel, as described above.

The opening in the channel is preferably smaller in width than the diameter of the tube to be held. This is particularly preferred in the case that the material of the base is resilient, allowing the opening in the channel to be temporarily widened to allow a tube to be inserted into the channel. The opening in the channel may be from 0.1 to 0.9 of the diameter of the channel, more preferably from 0.2 to 0.8, still more preferably from 0.3 to 0.7.

As noted, the base may be formed from a resilient material. This is particularly advantageous, as it allows the resilience to be used to urge the walls of the channel into contact with the tube after the tube has been inserted into the channel, to further grip the tube.

The material of the base is advantageously transparent, at least in the region of the channel, to allow the portion of the tube extending through the device to be viewed, as required.

The inner surface of the channel may be substantially smooth. Alternatively, the inner surface of the channel may be formed with one or more protrusions to grip and hold the tube.

The channel is formed in the base of the device, with its opening in the first or second major surface of the base. The channel extends through a portion of the thickness of the base. In particular, the ratio of the depth of the channel to the thickness of the base in the region of the channel may be from 1:1.05 to 1:5, more preferably up to 1:3, still more preferably up to 1:2. In one preferred embodiment, the thickness of the base is fractionally larger than the depth of the channel. In this way, the base comprises a relatively thin portion along the line of the channel, in turn rendering the base particularly flexible along the channel. This can allow a tube to be inserted into the channel more easily, before the device is secured to the skin of the patient. This also allows the position of the device along the tube to be adjusted, again before the base is secured to the skin of the patient. In this respect, the ratio of the depth of the channel to the thickness of the base in the region of the channel is from 1:1.05 to 1:1.3, more preferably from 1:1.1 to 1:1.2.

Alternatively, as described hereinbefore, the channel may be formed between two adjacent portions of the base. The adjacent portions are spaced apart along the length of the channel, to allow the device to flex along the length of the channel.

On a more general note, it has been found that, with the aforementioned arrangement with the base being flexible along the line of the channel, the action of securing the base to the skin of the patient urges the channel to close and thus grip the tube.

Accordingly, there is provided a device for supporting a tube or line, the device comprising a flexible base for securing to the skin of a patient; and a channel for holding the tube or line; wherein the action of securing the base to the skin of the patient urges the walls of the channel to grip the tube.

Preferred embodiments and features of the device are as hereinbefore described.

Embodiments of the present invention will now be described, by way of example only, having reference to the accompanying drawings, in which:

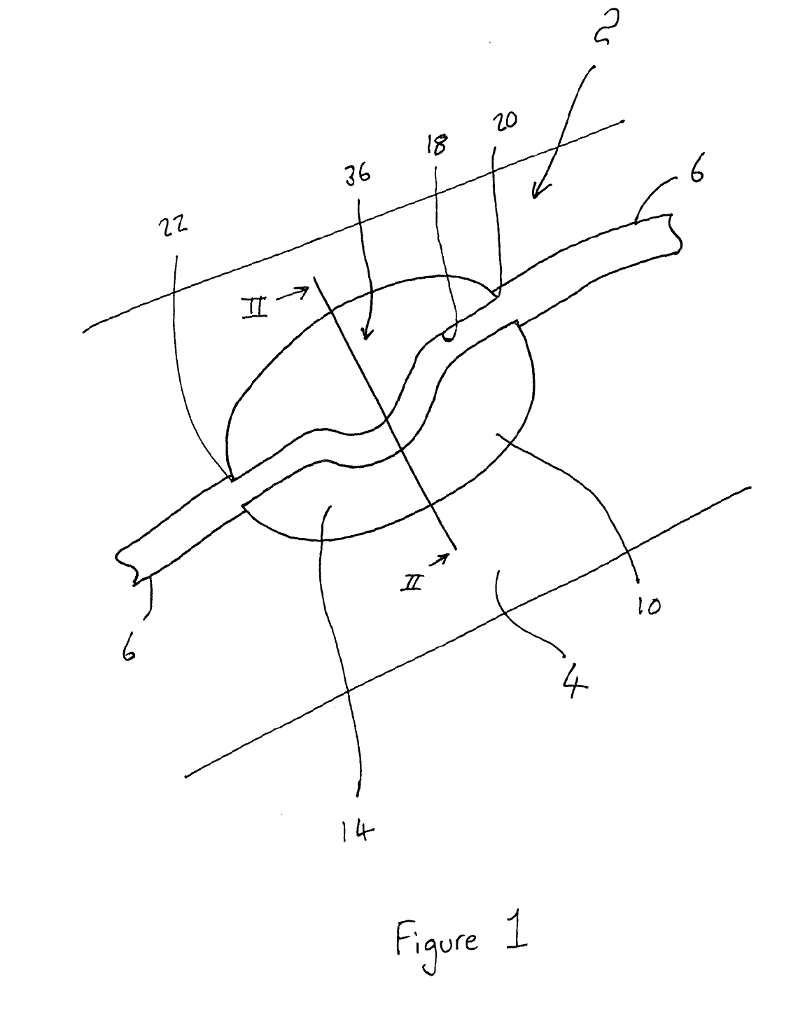

FIG. 1 is a perspective view of a device according to a first embodiment of the present invention attached to the skin of a patient and holding a tube;

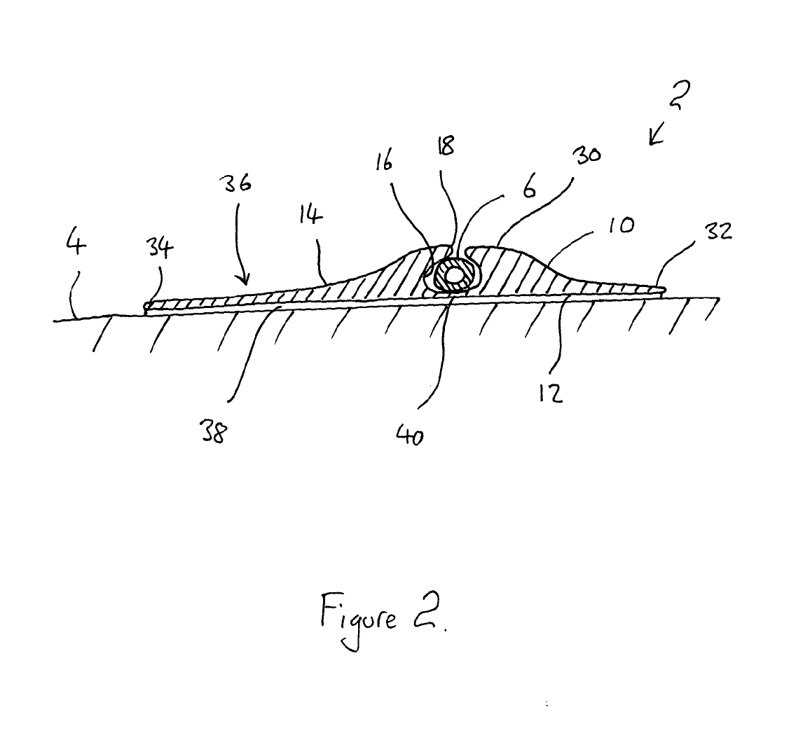

FIG. 2 is a cross-sectional view of the device of FIG. 1 along the line II-II;

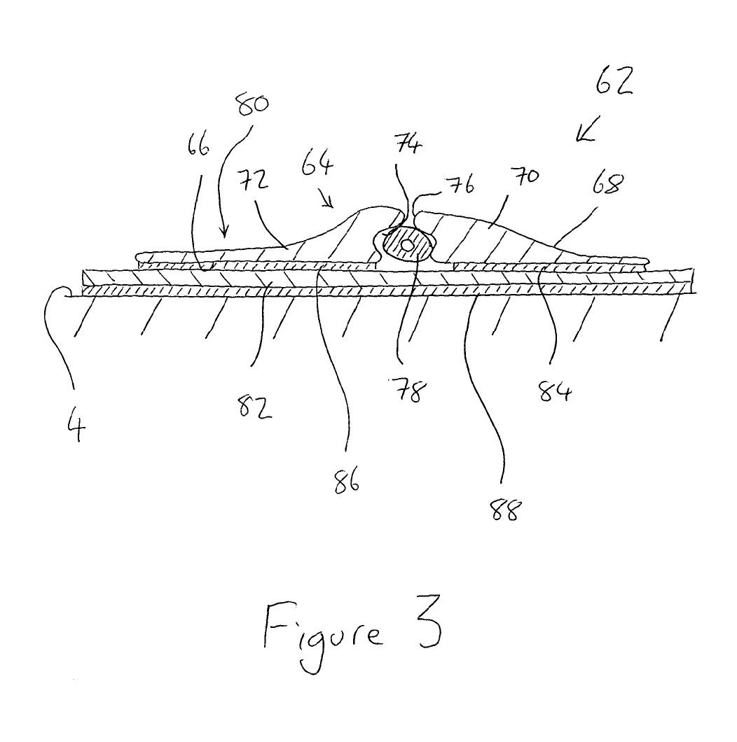

FIG. 3 is a cross-sectional view of an alternative embodiment of the device of FIG. 1;

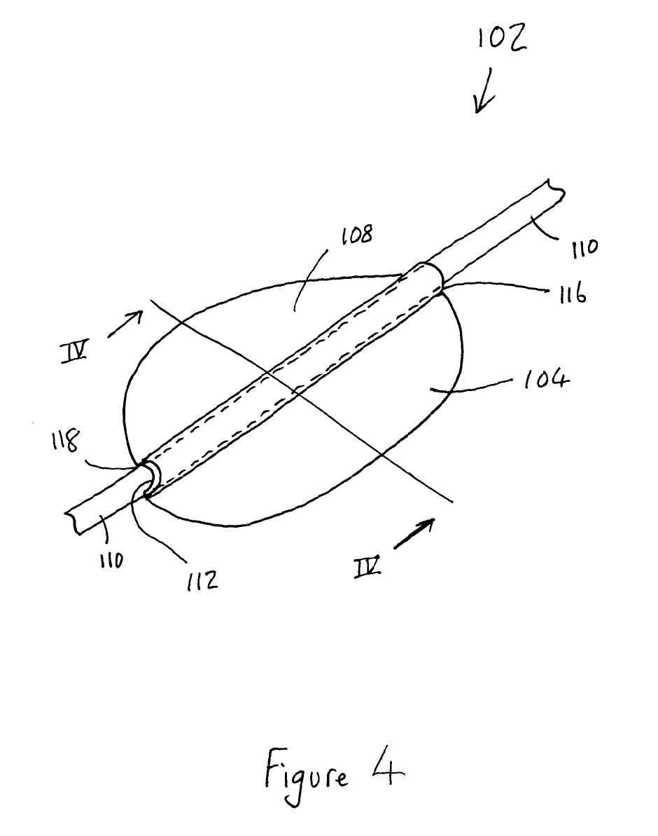

FIG. 4 is a perspective view of a device according to a further embodiment of the present invention holding a tube;

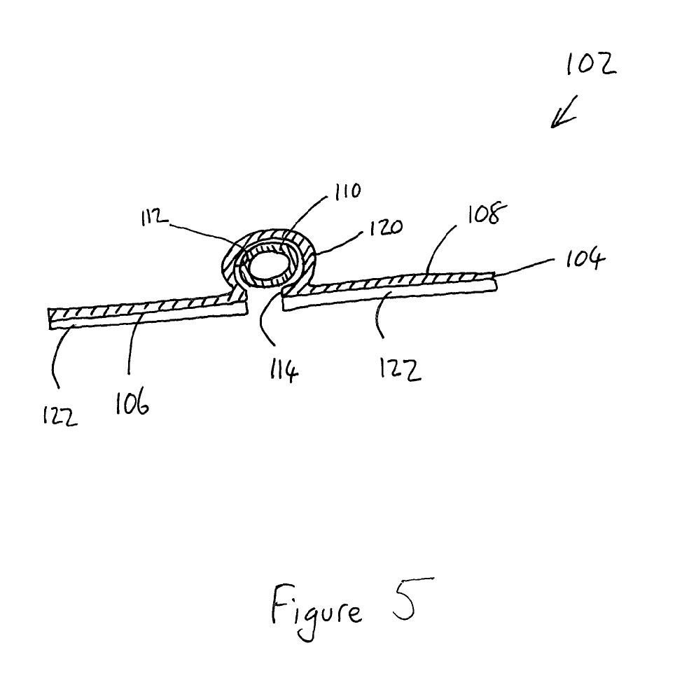

FIG. 5 is a cross-sectional view of the device of FIG. 3 along the line IV-IV; and

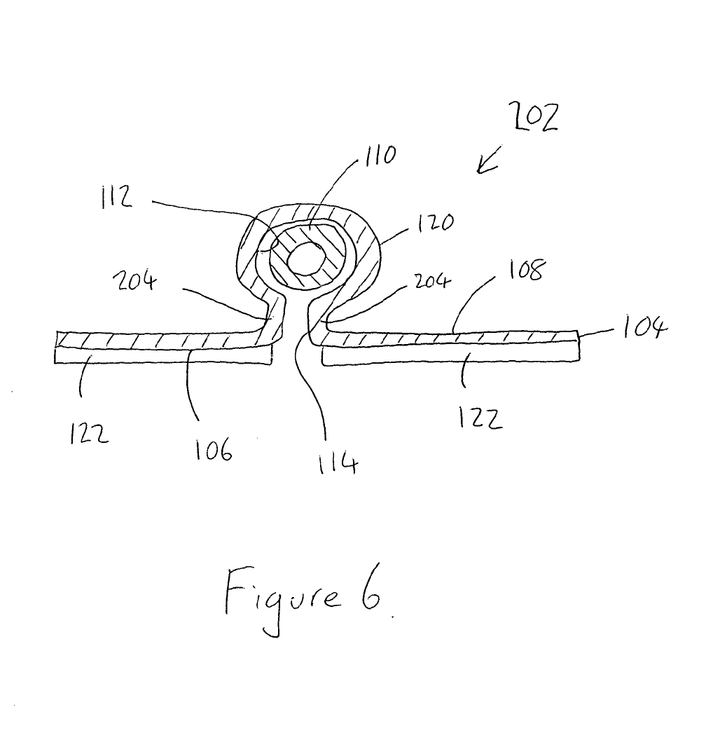

FIG. 6 is a cross-sectional view of an alternative embodiment of the device of FIG. 4.

Turning to FIG. 1, there is shown a device of a first embodiment, generally indicated as 2, adhered to the skin 4 of a patient. The device is shown secured to an arm of the patient. However, it is to be understood that the device may be secured to any part of the skin of the patient, as required for proper, safe and effective location of the tube being supported.

The device 2 is supporting a tube 6. The tube 6 is a line connected to a catheter inserted into a vein in the arm of the patient (not shown for clarity). However, it is to be understood that the device may be used to hold and support a wide range of medical tubes and lines required by a patient.

The device 2 comprises a generally oval base 10 formed from a flexible, resilient polyester. As shown in FIG. 2, the base has a first major surface 12, which in use faces the skin of the patient, and a second major surface 14 that in use is exposed, as shown in FIG. 1.

The base 10 has a channel 16 formed therein. The channel 16 has an opening 18 in the second major surface 14 of the base, as shown in FIG. 1. The channel 16 is generally circular in cross-section, having a diameter closely matched to that of the tube 6, such that once inserted into the channel the tube is firmly held by the resilient action of the base material.

The channel 16 has a first open end 20 in one end of the oval base and a second open end 22 in the second end of the base. As shown in FIG. 1, the channel 16 extends in a curved path through the base between its open ends 20, 22. In particular, the channel extends in a compound curve comprising three arc portions, such that the channel curves to one side of a straight line extending between its open ends 20, 22. In this way, the tube is securely held by the curved channel 16 and is not free to slide along inside the channel, for example under the normal movement of the patient.

As shown in FIG. 2, the base 10 has a thicker portion 30 in the region of the channel 16 and tapers to a lower thickness at its edge portions 32, 34. In this way, the base is flexible and is able to flex under the action of movement of the skin of the patient.

A portion of the second major surface 14, generally indicated as 36, is available for a medical practitioner to write notes on the device.

An adhesive layer 38 is provided on the first major surface, to adhere the base 10 to the skin 4 of the patient.

The region of the base 10 between the first major surface 12 and the channel 16 is indicated in FIG. 2 as 40. As shown, the region 40 is of considerably reduced thickness, compared with the other portions of the base 10. As a result, the base 10 is susceptible to flexing along the line of the channel 16, allowing the opening 18 to be widened to accept the tube 6. Further, adhering the base 10 to the skin 4 of the patient has the effect of urging the walls of channel 16 together and closing the opening 18, thus closing the channel about the tube 6, further gripping and securing the tube.

Turning to FIG. 3, there is shown a cross-sectional view of an alternative embodiment of the device of FIGS. 1 and 2, generally indicated as 62, adhered to the skin 4 of a patient. The device may be secured to any part of the skin of the patient, as required for proper, safe and effective location of the tube being supported.

The device 62 comprises a generally oval base 64 formed from a flexible, resilient polyester. As shown in FIG. 3, the base has a first major surface 66, which in use faces the skin of the patient, and a second major surface 68 that in use is exposed, as shown in FIG. 3. The base 64 comprises a first base portion 70 and a second base portion 72. The first and second base portions 70, 72 are spaced apart to form a channel 74 therebetween, again as shown in FIG. 3. The channel 74 has an opening 76 in the second major surface 68 of the base. The channel 16 is generally circular in cross-section, having a diameter closely matched to that of a tube 78, such that once inserted into the channel the tube 78 is firmly held by the resilient action of the base material.

The channel 74 may extend in any shape through the base 64. The embodiment shown in FIG. 3 is particularly suitable for use with a channel having a curved or arcuate form, such as the channel shown in FIG. 1 and described above.

As shown in FIG. 3, the base 64 has a thicker portion in the region of the channel 74 and tapers to a lower thickness at its edge portions. In this way, the base is flexible and is able to flex under the action of movement of the skin of the patient.

A portion of the second major surface 68 of the second portion of the base 72, generally indicated as 80, is available for a medical practitioner to write notes on the device.

The device 62 further comprises a layer of flexible material 82, such as a resilient polyester, which in use extends between the first and second base portions 70, 72 and the skin 4 of the subject. Each base portion 70, 72 is secured to the flexible layer 82 by a respective layer of adhesive 84, 86. As a result, the base 64 is susceptible to flexing along the line of the channel 74, allowing the opening 76 to be widened to accept the tube 78. Further, adhering the base 64 to the skin 4 of the patient has the effect of urging the walls of channel 74 together and closing the opening 76, thus closing the first and second base portions 70, 72 and the channel about the tube 78, further gripping and securing the tube.

The device is secured to the skin 4 of the subject by an adhesive layer 88.

As shown in FIG. 3, the flexible layer 82 extends beyond the edges of the first and second base portions 70, 72 and prevents the skin 4 being abraded by the edges of the base portions.

Turning to FIG. 4, there is shown a perspective view of a device of a second embodiment of the present invention, generally indicated as 102. The device comprises a generally oval base 104 of a flexible, transparent, resilient polyester.

As shown in FIG. 5, the base has a first major surface 106, which in use faces the skin of the patient, and a second major surface 108 that in use is exposed, as shown in FIG. 4.

The device 102 is holding a tube 110.

The base 104 has a channel 112 formed therein. The channel 112 has an opening 114 in the first major surface 106 of the base, as shown in FIG. 5. The channel 112 is generally circular in cross-section, having a diameter closely matched to that of the tube 110, such that once inserted into the channel the tube is firmly held by the resilient action of the base material.

The channel 112 extends substantially linearly across the base 104 from opposing edges and has open ends 116 and 118 in the opposing edges of the base.

As shown in FIG. 5, the base 104 is of generally uniform thickness, with the exception that the base is thicker in the immediate region of the channel 112, so as to form the channel with a wall 120 substantially the same thickness as the remaining portions of the base.

An adhesive layer 122 is provided on the first major surface 106 of the base on either side of the channel 112, for securing the device to the skin of the patient.

In use, the tube 110 is inserted into the channel 112 through the opening 114. To assist this, the base 104 may be flexed, to widen the opening 114. Once the tube is located in the channel, the device is positioned along the tube. Once in position, the device 102 is adhered to the skin of the patient, such that the opening 114 is facing the skin of the patient, holding the tube in place in the channel 112. Further, the action of adhering the base to the skin of the patient urges the wall 120 of the channel 112 into closer contact with the tube 110, thus further gripping the tube.

Turning to FIG. 6, there is shown an alternative embodiment of the device shown in FIGS. 4 and 5. The device of FIG. 6, generally indicated as 202, is generally of the same form and construction as the device of FIGS. 4 and 5. Accordingly, components common to the two devices have been indicated using the same reference numerals, as described above. In the device of FIG. 6, the base 104 is formed with wall members 204 extending upwards from the base, as viewed in the figure. The wall members 204 extend either side of the opening 114 in the base, such that the tube 110 when present in the channel 112 is held at a distance from the second major surface 108 of the base. In this way, the tube 110 is held away from the skin of the subject when the device is in use. This in turn reduces the tendency for the tube to rub against and abrade the skin of the subject in the region of the device.

1. A device for supporting a tube, the device comprising a flexible base for securing to the skin of a patient and having a first major surface for opposing the skin of the patient and a second major surface opposite the first, the base comprising a channel extending therethrough, the channel opening in the first or the second major surface of the base.

2. The device according to claim 1, wherein the channel is formed with its opening in the second major surface of the skin.

3. The device according to claim 1, wherein the channel is formed in the first major surface of the skin.

4. The device according to claim 1, wherein the material of the base is resilient.

5. The device according to claim 1, wherein the base is formed from a polymer.

6. The device according to claim 1, wherein the material of the device exhibits significant friction with the material of the tube to be held.

7. The device according to claim 1, wherein the base has a thickness of from 1.05 to 3 times the diameter of the tube to be held.

8. The device according to claim 1, wherein the base has a first thickness in the region of the channel and a second thickness at the edge portions of the base, the first thickness being greater than the second thickness.

9. The device according to claim 1, wherein the base has a thickness of from 0.5 to 15 mm.

10. The device according to claim 1, wherein the base is substantially circular or oval.

11. The device according to claim 1, wherein the base has an ornate shape.

12. The device according to claim 1, wherein the channel extends across the entire width of the base.

13. The device according to claim 1, wherein the channel has a length of from 5 to 25 times the diameter of the tube to be held.

14. The device according to claim 1, wherein the base is formed as a single component.

15. The device according to claim 1, wherein the base comprises a first base portion and a second base portion, the channel being defined between the first and second base portions.

16. The device according to claim 15, wherein the first and second base portions are joined by a flexible member.

17. The device according to claim 16, wherein the flexible member is at least coterminous with the first and second base portions.

18. The device according to claim 17, wherein the flexible member extends beyond the edges of the first and second base portions.

19. The device according to claim 1, wherein the device further comprises a layer of flexible material extending across the first major surface of the base.

20. The device according to claim 19, wherein the layer of flexible material extends beyond the edges of the base.

21. The device according to claim 1, wherein the second major surface of the base comprises a region for displaying information.

22. The device according to claim 1, wherein the base has two or more channels formed therein.

23. The device according to claim 1, wherein the channel is disposed to hold the tube out of contact with the skin of the subject when the device is in use.

24. The device according to claim 1, wherein the channel is substantially linear.

25. The device according to claim 1, wherein the channel extends through the base in a curved pattern.

26. The device according to claim 25, wherein the curve of the channel consists of a single arc.

27. The device according to claim 25, wherein the curve of the channel comprises a plurality of arcs.

28. The device according to claim 27, wherein the curve extends to both sides of a line joining the open ends of the channel.

29. The device according to claim 1, wherein the walls of the channel extend substantially straight from the base of the channel to the opening.

30. The device according to claim 1, wherein the walls of the channel are curved between the base of the channel and the opening.

31. The device according to claim 30, wherein the cross-section of the interior of the channel is generally circular to match the tube to be held.

32. The device according to claim 1, wherein the inner surface of the channel is substantially smooth.

33. The device according to claim 1, wherein the inner surface of the channel is provided with projections to grip the tube to be held.

34. The device according to claim 1, wherein the action of applying the device to the skin of the subject urges the channel to close and grip the tube to be held.

35. A method of holding a tube at the skin of a subject, comprising securing a device according to claim 1 to the skin of a subject and inserting a tube within the channel of the device.
